(19) United States
(12) Patent Application Publication
(10) Pub. No.: US 2013/0035488 A1
(43) Pub. Date: Feb. 7, 2013

(54) Stepwise Process for the Production of Alkaloid Salts

(71) Applicants: Bradley R. Jones, Westerville, OH (US); Joseph P. Haar, JR., Edwardsville, IL (US); Kevin R. Roesch, Godfrey, IL (US); Leslie L. Vanderkolk, Pittsburgh, PA (US)

(72) Inventors: Bradley R. Jones, Westerville, OH (US); Joseph P. Haar, JR., Edwardsville, IL (US); Kevin R. Roesch, Godfrey, IL (US); Leslie L. Vanderkolk, Pittsburgh, PA (US)

(73) Assignee: Mallinckrodt LLC, Hazelwood, MO (US)

(21) Appl. No.: 13/563,822

(22) Filed: Aug. 1, 2012

Related U.S. Application Data
(60) Provisional application No. 61/514,088, filed on Aug. 2, 2011.

(51) Int. Cl. C07D 489/02

(52) U.S. Cl. 546/45

(57) ABSTRACT

The present invention provides to an improved process for preparing alkaloid salts. In particular, the process comprises a stepwise addition of an acid to an alkaloid chosen from hydrocodone, codeine, and dihydrocodeine to form a flowable mixture of the alkaloid salt.

### CROSS REFERENCE TO RELATED APPLICATIONS

This application claims the benefit of U.S. Provisional Application No. 61/514,088 filed Aug. 2, 2011, which is incorporated herein in its entirety.

### FIELD OF THE INVENTION

The present invention relates to an improved process for preparing alkaloid salts. In particular, it relates to a process for preparing alkaloid salts through stepwise addition of an acid to an alkaloid.

### BACKGROUND OF THE INVENTION

Alkaloids are important compounds for a variety of pharmaceutical uses. For pharmaceuticals, alkaloids are useful in their salt form. Alkaloid salts crystallize well, in contrast to their free base forms. Crystallization provides for greater consistency in the properties of the alkaloid salt. Salts are also water soluble, an important property in administering the pharmaceutical. Various salt forms can modulate a pharmaceutical's characteristics to provided better bioavailability, stability, and patient compliance. Thus, formation of a desired salt is a crucial part of drug development. (C. G. Wermuth and P. H. Stahl, Handbook of Pharmaceutical Salts: Properties, Selection and Use, 1-7 (Wiley-VCH, Weinheim, Germany, 2002).

Salt formation, and the resulting salt forms, however, can be unpredictable. Formation of the crystalline alkaloid salt may occur in such a way that solvents become trapped in the crystal network, which is problematic because the solvent levels of pharmaceuticals are regulated. Moreover, trapped solvents can result in a slurry of the alkaloid salts that is not flowable. A mixture that is incapable of flow presents a number of related problems including labor intensive removal of the products and higher levels of exposure of employees to hazardous solvents.

Attempts to form alkaloid salts in a manner which results in a flowable mixture through changing the temperatures, running the reactions in a more dilute system, and/or changing the solvents have failed to produce flowable mixtures. Thus, there is a need for a process for producing alkaloid salts in a flowable mixture.

### SUMMARY OF THE INVENTION

The present invention relates to an improved process for the production of alkaloid salts. The alkaloids of the invention may comprise hydrocodone, codeine, or dihydrocodeine. Alkaloid salts can be formed by reaction with a variety of acids to form the alkaloid salts, preferably, those suitable for pharmaceutical uses such as bitartrate, bromide, citrate, chloride, mesylate, maleate and phosphate salts.

The process comprises the stepwise addition of the acid to the alkaloid. Stepwise addition results in a crystalline form of the salt that is a flowable mixture.

### DETAILED DESCRIPTION OF THE INVENTION

Briefly, therefore, the present invention provides a process for the production of alkaloid salts comprising a stepwise addition of an acid to the alkaloid to form a flowable mixture of the crystalline alkaloid salts. By flowable, it is meant that the mixture exhibits properties of fluid movement and exhibits movement with the force of gravity such that it is capable of being poured.

In general, the alkaloids that can be used in the process are those which are known in the art for reacting with acids to produce the alkaloid salts in mixtures which are not flowable. The alkaloids may be chosen from hydrocodone, codeine, or dihydrocodeine, and isomers thereof.

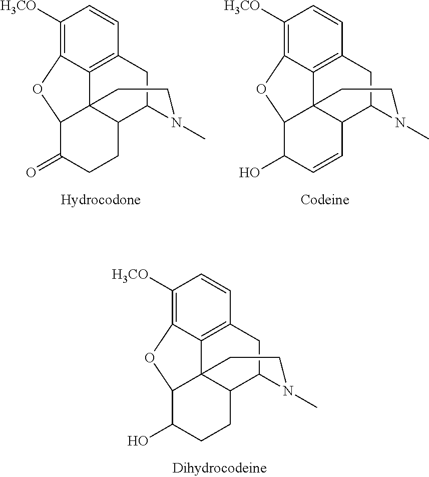 [SMILES: COc1ccc2c3c1OC1C(=O)CCC4C(C2)N(C)CCC314 ; COc1ccc2c3c1OC1C(O)C=CC4C(C2)N(C)CCC341 ; COc1ccc2c3c1OC1C(O)CCC4C(C2)N(C)CCC341]

The amine groups of the alkaloids are weakly basic, thus, they can form a salt with the addition of a Bronsted acid. Bronsted acids are capable of donating a proton to the amine group of the alkaloid. The resulting charged species is stabilized by a counterion to form the alkaloid salt. Preferably, the acid reacts with the alkaloid to produce a pharmaceutically acceptable salt. Non-limiting examples of acids are acetic, adipic, alginic, ascorbic, aspartatic, benzenesulfonic, benzoic, camphoric, capric, caprylic, carbonic, citric, cyclamic, dodecylsulfuric, ethanesulfonic, ethane-1,2-disulfonic, fumaric, galactaric, gentisic, glucoheptanoic, gluconic, glucuronic, glutamic, glutaric, glycolic, glycerophosphoric, hippuric, hydrochloric, hydrobromic, hydroxy, isobutyric, lactic, lactobioni, lauric, maleicm, malonic, methanesulfonic, malic, naphthalene-1,5-disulfonic, naphthalene-2-sulfonic, 2-napthoic 1-hydroxy, nicotinic, oleic, orotic, 2-oxo glutaric, oxalic, palmitic, pamoic, propionic, pyroglutamic, phosphoric, sebacic, succinic, sulfuric, tartaric, thiocyanic, p-toluenesulfonic, stearic acid, and mixtures thereof. More preferably, the acids is chosen from hydrochloric, hydrobromic, citric, phosphoric, and tartaric acid. In a preferred embodiment, the acid is tartaric acid.

The alkaloid is typically dissolved or suspended in a solvent system prior to introduction of the acid. The solvent system may comprise an organic solvent or a mixture of an organic solvent and water. Acceptable organic solvents include alkane and substituted alkane solvents (including cycloalkanes) alcohol solvents, halogenated solvents, aromatic hydrocarbons, esters, ethers, ketones, and combinations thereof. Non-limiting examples of specific organic solvents acetonitrile, acetone, allyl alcohol, benzene, butyl acetate, chlorobenzene, chloroform, chloromethane, cyclohexane, cyclopentane, dichloromethane, dichloroethane, diethyl ether, dimethyl sulfonic acid, dioxane, ethanol, ethyl acetate, ethylene dichloride, ethylene bromide, fluorobenzene, heptane, hexane, isobutylmethylketone, isopropanol, isopropyl acetate, methanol, methylene bromide, methylene chloride, methyl iodide, methylethylketone, methyltetrahydrofuran, pentyl acetate, propanol, n-propyl acetate, tetrahydrofuran, tetrachloroethane, toluene, tricholorethane, water, xylene, and combinations thereof. In some embodiments, the organic solvent is ethanol. In a preferred embodiment, the organic solvent is a mixture of about 95% ethanol and about 5% methanol.

In some embodiments, the solvent system comprises water. In embodiments where the solvent system comprises water, it is preferable that the organic solvent is miscible with water. The amount of water in the solvent system may vary between 0 and 50% of the solvent system. In some embodiments, the percent of water ranges between 5% and 20%. In other embodiments, the amount of water ranges between 10% and 13%. In a preferred embodiment, the amount of water in the solvent system is 10%.

The amount of the solvent system may vary with in relation to the amount of alkaloid. In some embodiments, the molarity of the mixture of the alkaloid and the solvent system ranges from about 0.1 moles/liter to about 0.5 moles/liter. In other embodiments, the molarity of the mixture of the alkaloid and the solvent system ranges from about 0.15 moles/liter and 0.25 moles/liter. In a preferred embodiment, the molarity of the mixture of the alkaloid and the solvent system is about 0.2 moles/liter.

The addition of acid to the alkaloid is generally accompanied by mixing of the resulting reaction mixture. Mixing may be performed by any means known in the art including manual and automatic mixing. In an exemplary embodiment, mixing is provided by a blade set to mix the reagents at about 200 RPM.

The acid is added to the alkaloid in a stepwise manner to form the alkaloid salt in a flowable mixture. By stepwise, it is meant, that portions of the acid are introduced to the alkaloid in discrete amounts at intervals to reach the desired total of the acid. In some embodiments, the first addition of the acid to the alkaloid is about 40 to about 60% of the total amount of acid to be added. In alternate embodiments, the first addition of the acid to the alkaloid is about 50% of the total to be added. In yet another embodiment, the first addition of the acid to the alkaloid is about 40% of the total acid to be added to the mixture.

The first addition is followed by subsequent smaller additions. In some embodiments, subsequent additions may vary between about 5% and about 25% of the total acid to be added to the mixture. In other embodiments, the subsequent additions may vary between about 10% and about 15% of the total acid to be added to the mixture. In a further embodiment, the subsequent additions may vary between about 15% and about 20% of the total acid to be added to the mixture. In one preferred embodiment, the subsequent additions may vary between about 10% and about 12% of the total acid to be added to the mixture. Depending on the amounts added in each addition, there may be three or more additions of acid to the alkaloid.

The amount of time between additions of the acid may also vary. The amount of time that each sequential addition of the acid is allowed to stir between additions may vary from about 0.1 hours to more than about 3 hours. In another embodiment, the amount of time that each sequential addition of the acid is allowed to stir between additions can vary from about 0.5 hours and about 2 hours. In a preferred embodiment, the amount of time that each sequential addition of the acid is allowed to stir between additions varies from about 0.5 hours to about 1 hour.

The mole-to-mole ratio of the basic amine present in the alkaloid to the acid can and will vary from about 1:0.5 to about 1:1.5. In another embodiment, the mole-to-mole ratio of the basic amine present in the alkaloid to the acid may vary from about 1:1 to about 1:1.4. In other embodiments, the mole-to-mole ratio of the basic amine present in the alkaloid to the acid may vary from about 1:0.8 to about 1:1, from about 1:0.9 to about 1:1.1, from about 1:0.9 to about 1:1.2, from about 1:1 to about 1:1.2, from about 1:1.2 to about 1:1.3, from about 1:1.3 to about 1.5, and from about 1:1.4 to about 1:1.5. In an exemplary embodiment, the ratio of the basic amine present in the alkaloid to the acid is 1:1.1.

The temperature of the reaction may also vary. In some embodiments, the reaction can be conducted at a temperature ranging from about 25° C. to about 80° C. In another embodiment, the reaction is carried out at a temperature ranging between about 40° C. and about 75° C. In other embodiments, the temperature ranges from about 20° C. to about 30° C., from about 25° C. to about 35° C., from about 30° C. to about 40° C., from about 35° C. to about 45° C., from about 40° C. to about 50° C., from about 55° C. to about 65° C., from about 60° C. to about 70° C., from about 65° C. to about 75° C., and from about 70° C. to about 80° C. In an exemplary embodiment, the reaction is carried out at 70° C.

Where the reaction is conducted above room temperature, the reaction is generally stirred without heating after the stepwise addition for a sufficient time for the reaction to cool. In other embodiments, the mixture is cooled below room temperature after the stepwise addition. In various embodiments, crystallization of the alkaloid occurs with cooling, while in other embodiments, crystallization occurs upon addition of the acid to the alkaloid.

The reaction may further comprise one or more additional steps including filtration and removal of solvent. Filtration can be performed by any known means and may be directed at various products. Removal of solvent may be provided by decanting, draining, vacuum, distilling, drying, or any other method.

The process results in a flowable mixture of the crystalline alkaloid salts. The flowable mixture exhibits properties of a liquid in that it is movable under the force of gravity such that it can be poured or transferred by pump from one vessel to another. The crystalline alkaloid salts further exhibit desired characteristics for pharmaceutical use, including water content, solvent content, bulk density, and distribution of particle size.

Having described the invention in detail, it will be apparent that modifications and variations are possible without departing from the scope of the invention defined in the appended claims.

### EXAMPLES

The following examples detail various embodiments of the process described and are not meant to be limiting of the process described above.

### Example 1

### Comparative Example: Addition in Two Portions

Tartaric acid (½ mol) was added to hydrocodone alkaloid (1 mol) in 86% ethanol, 10% water, and 4% methanol. The mixture was stirred and heated to 74° C. A mixture of tartaric acid (½ mol) in 86% ethanol, 10% water, and 4% methanol was added. The resulting solution was stirred for approximately 1 hour then cooled from 74° C. to 10° C. over 4 hours. Stirring was stopped as the product crystallized and resulted in a non-flowable mixture.

### Example 2

### Stepwise Addition of Tartaric Acid to Hydrocodone Bitartrate

Hydrocodone alkaloid (1 mol. equiv) was added to a solution of 10% water, 85.5% ethanol, and 4.5% methanol to make a mixture of approximately 80 g/L. The temperature of the mixture was controlled by means of a heat/cool module programmed to maintain 53° C. and an automatic stirrer was set to 200 RPM (rotations per minute). Tartaric acid (0.4 mol equiv.) was added to the mixture and allowed to stir for 10 minutes. A second portion of tartaric acid (0.1 mol equiv.) was added to the reaction mixture and allowed to stir for 0.5 hr. A third portion of tartaric acid (0.1 mol equiv.) was added to the reaction mixture and allowed to stir for 2 hr. A fourth portion of tartaric acid (0.1 mol equiv.) was added to the reaction mixture and allowed to stir for 1 hr. A fifth portion of tartaric acid (0.1 mol equiv.) was added to the reaction mixture and allowed to stir for 45 min. A sixth portion of tartaric acid (0.1 mol equiv.) was added to the reaction mixture and allowed to stir for 45 min. The mixture remained flowable at 53° C., and was cooled to 10° C. The mixture remained flowable at 10° C.

What is claimed is:

1. A process for the production of alkaloid salts, the process comprising a stepwise addition of an acid to an alkaloid, wherein the alkaloid is chosen from hydrocodone, codeine, or dihydrocodeine, and the process results in a flowable mixture of the alkaloid salt.

2. The process of claim 1, wherein the acid is chosen from acetic, adipic, alginic, ascorbic, aspartatic, benzenesulfonic, benzoic, camphoric, capric, caprylic, carbonic, citric, cyclamic, dodecylsulfuric, ethanesulfonic, ethane-1,2-disulfonic, fumaric, galactaric, gentisic, glucoheptanoic, gluconic, glucuronic, glutamic, glutaric, glycolic, glycerophosphoric, hippuric, hydrochloric, hydrobromic, hydroxy, isobutyric, lactic, lactobioni, lauric, maleicm, malonic, methanesulfonic, malic, naphthalene-1,5-disulfonic, naphthalene-2-sulfonic, 2-napthoic 1-hydroxy, nicotinic, oleic, orotic, 2-oxo glutaric, oxalic, palmitic pamoic, propionic, pyroglutamic, phosphoric, sebacic, succinic, sulfuric, tartaric, and thiocyanic, p-toluenesulfonic acids, and stearic acid.

3. The process of claim 1, wherein the process further comprises a solvent system.

4. The process of claim 3, wherein the solvent system comprises an organic solvent.

5. The process of claim 4, wherein organic solvent is chosen from methanol, ethanol and isopropanol.

6. The process of claim 3, wherein the solvent system further comprises water.

7. The process of claim 6, wherein water comprises about 10 to about 13% of the total solvent system.

8. The process of claim 1, wherein the stepwise addition comprises a first portion of about 40% to about 60% of the acid, and subsequent portions range from about 10% to about 20% of the acid.

9. The process of claim 1, wherein the stepwise addition comprises a first portion of about 60% of the acid with subsequent portions of about 10% of the acid.

10. The process of claim 1, wherein the stepwise addition comprises a first portion of about 55% of the acid with subsequent portions of about 10% of the acid.

11. The process of claim 1, wherein the stepwise addition comprises a first portion of about 50% of the acid with subsequent portions of about 10% of the acid.

12. The process of claim 1, wherein the process is conducted at a temperature ranging from about 50° C. to about 70° C.

13. The process of claim 12, wherein the reaction mixture is cooled to a temperature below about 25° C. to form a flowable alkaloid salt mixture.

14. The process of claim 1, wherein the ratio between the amine group of the alkaloid and the acid is about 1:1.1, respectively.

15. The process of claim 1, wherein the alkaloid salt is produced in a yield above about 90%.

16. The process of claim 3, wherein the acid is tartaric acid, the alkaloid is chosen from hydrocodone and dihydrocodeine, and the solvent system is comprised of methanol, ethanol, and water.

17. The process of claim 3, wherein the acid is phosphoric acid, the alkaloid is codeine, and the solvent system is comprised of methanol, ethanol, and water.

18. A process for the production of hydrocodone bitartrate wherein the process comprises the following steps:
a. adding about 0.4 equivalents of tartaric acid to a mixture of 1 equivalent of hydrocodone in about 85% ethanol, 5% methanol, and about 10% water, and stirring the mixture for about 1 hour;
b. adding about 0.1 equivalents of tartaric acid to the mixture and stirring for about 1 hour; and
c. repeating step (b) until about 1.1 equivalents of tartaric acid have been added to the reaction to result in a flowable mixture of hydrocodone bitartrate.
